(19) United States
(12) Patent Application Publication
(10) Pub. No.: US 2024/0245940 A1
(43) Pub. Date: Jul. 25, 2024

(54) PROTECTIVE FACE MASK

(71) Applicant: ESG Supplies LTD, London (GB)

(72) Inventors: Robert David McIntosh, Marsworth (GB); David Ronald Whitworth, Dubai (UA); Wayne Christopher Connor, Dubai (UA); Anthony John Rudd, Teignmouth, Devon (GB); Roderick Bruce Crawford, Dubai (UA); Sandeep Kumar Chintala, Greater London (GB)

(21) Appl. No.: 18/560,649

(22) PCT Filed: May 13, 2022
(86) PCT No.: PCT/EP2022/063109

§ 371 Date: Nov. 13, 2023

Related U.S. Application Data
(60) Provisional application No. 63/188,433, filed on May 13, 2021.

(51) Int. Cl. A62B 18/04; A61L 9/20; A62B 18/08; A62B 18/10; A62B 23/02

(52) U.S. Cl. CPC A62B 18/045; A61L 9/20; A62B 18/08; A62B 23/02; A61L 2209/111; A61L 2209/12; A61L 2209/14; A62B 18/10

(57) ABSTRACT

A smart face mask protects a user from harmful contaminants found in the user's environment. The face mask includes inlet and outlet filters to not only protect the user, but also other people within a vicinity of the user. The inlet and outlet filters include fans that promote proper air flow into and out of the mask. Air respiratory sensors provide accurate measurements of possible air contaminants. Sensors also collect data that is used to control and change fan speed operations. A mobile application controlled by the user is used to control operation of the face mask during use. Communications via short range wireless network 150s are provided between people via microphones, speakers, or earpieces. The face mask may be worn in shared spaces, indoor environments, or outdoor environments.

### FIELD OF THE DISCLOSURE

The field of the disclosure relates generally to personal protective equipment, and more particularly, to personal protective masks having integration of user a user's biometric data or personal data associated with one or more of the user's vital signs.

### BACKGROUND

Personal protective equipment (“PPE”) is a class of equipment used to protect the health of the PPE user and those proximate the person wearing PPE. Because PPE covers the user's nose and mouth, when using PPE, the user's health is less likely to be negatively impacted by the ambient conditions and by those who are not wearing PPE equipment who come in close contact with the PPE user. Alternatively, the health of those who come in close contact with the PPE user are less likely to be negatively impacted by someone who is utilizing PPE.

PPE can comprise simple cloth or paper masks that cover the user's nose and mouth or face shields that cover the user's eyes, nose and mouth. More extensive PPE comprise face masks that cover the user's face and form a seal against the users face, creating a ‘breathing chamber’ for the user. This form of PPE includes means for both breathing in ambient air and flowing respired air are back into the environment. Typically, the breathing means is included on the front of the mask, proximate the user's nose and mouth. The breathing means adds significant weight to the front of the PPE and during long periods of use, the weight of the breathing means can produce discomfort in the user's neck.

Because the respired air is not filtered before being released into the ambient environment, the respired air may include microorganisms that could negatively impact the health of a person located proximate the mask user.

Additionally, because the breathing means is located near the user's mouth, communication with the user can be hindered by the breathing means. The breathing means may muffle the user's voice and speech. Such communication challenges are particularly problematic when the PPE is worn in an operating room setting or in a situation where effective communication between parties is critical.

Ambient conditions and user's personal health can negatively impact the user's ability to see through the face mask. Humid environments or an increase in the user's body temperature, or respiration rate can cause the mask to undesirably ‘fog’ and impede the user's vision through the mask.

There is a need for a PPE that solves the foregoing shortcomings associated with current PPE.

### BRIEF SUMMARY OF THE DISCLOSURE

The present disclosure is directed to a protective face mask assembly. The protective face mask assembly includes a housing sized and configured to fit onto a head of a user, the housing having a lateral left portion and a lateral right portion; a visor supported by a frame, the visor and frame forming a sealed structure; a chamber defined by the housing and the frame; a plurality of first openings formed along the left lateral portion configured as an air inlet; and, a plurality of second openings formed along the right lateral portion configured as an air outlet. The plurality of first openings and the plurality of second openings are in fluid communication with the chamber.

### BRIEF DESCRIPTION OF THE DRAWINGS

This disclosure is illustrated by way of example and not by way of limitation in the accompanying figure(s). The figure(s) may, alone or in combination, illustrate one or more embodiments of the disclosure. Elements illustrated in the figure(s) are not necessarily drawn to scale. Reference labels may be repeated among the figures to indicate corresponding or analogous elements.

The detailed description makes reference to the accompanying figures in which:

FIG. 1 illustrates a schematic representation of a functional block diagram of a computer system in accordance with one or more embodiments of the disclosed disclosure;

FIG. 2 illustrates a perspective view an exemplary face mask in accordance with one or more embodiments of the present disclosure;

FIG. 3 illustrates a side view of the face mask of FIG. 1 in a closed position.

FIG. 4 illustrates a side view of the face mask of FIG. 1 in an open position.

FIG. 5 illustrates a bottom view of the face mask of FIG. 1 in an open position.

FIG. 6 illustrates a rear view of the face mask of FIG. 1 in an open position.

FIG. 7A illustrates an exemplary airflow overview in accordance with one or more embodiments of the disclosed disclosure;

FIG. 7B illustrates an exemplary airflow overview in accordance with one or more embodiments of the disclosed disclosure;

FIG. 7C illustrates an exemplary airflow overview in accordance with one or more embodiments of the disclosed disclosure; and,

FIG. 8 illustrates a schematic representation of a controller system in accordance with one or more embodiments of the present disclosure;

### DETAILED DESCRIPTION

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

### Acronyms, Abbreviations and Definitions

WSI Wireless System Integration
ALS Ambient Light Sensor
APR Air Purifying Respirator
ASIC Application Specific IC
BLE Bluetooth Low Energy
BT Bluetooth
cfm cubic feet meter
e.g. exempli gratia (for example)
etc. et cetera (and so on)
I2C Inter Integrated Circuit
I2S Inter IC Sound
IC Integrated Circuit
IEC International Electrotechnical Commission
LED Light Emitting Diode
Mbps Mega bit per second
MCU Microcontroller unit
NVM Non-Volatile Memory
ODM Original Device Manufacturer
PAPR Powered APR
PCB Printed Circuit Board
PCBA PCB Assembly
PIFA Planar Inverted F Antenna
PMU Power Management Unit
RGB Red Green Blue
SIM Subscriber Identity Module
SW Software
TBD To Be Defined
UI User Interface
USB Universal Serial Bus
USB PD USB Power Delivery

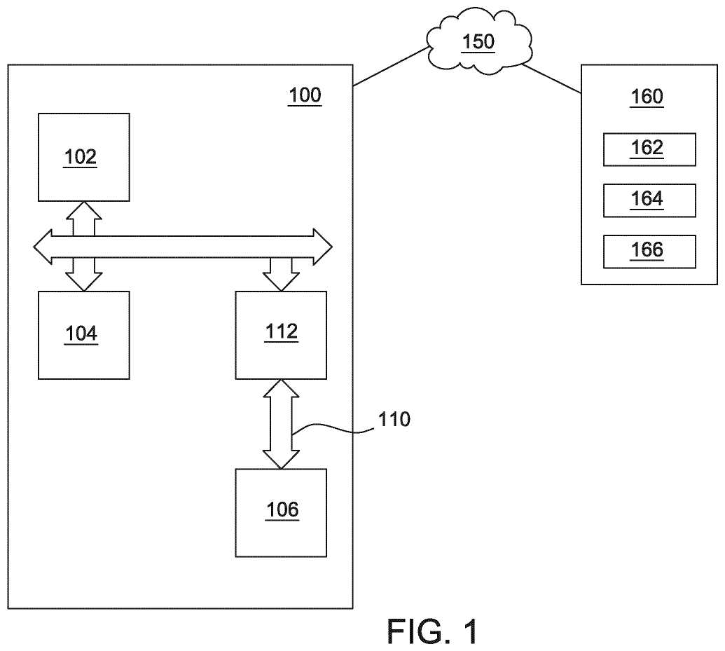

FIG. 1 illustrates a schematic representation of a functional block diagram of a computing system 100 and a network 150 of a face mask 200. The functional descriptions of the computing system 100 can be implemented in hardware, software or some combination thereof.

As shown in FIG. 1, the computing system 100 includes a processor 102, a memory 104 and one or more input/output (I/O) devices 106 in commutatively coupled to one another. In some embodiments, components of the computing system 100 are connected by one or more computer buses (108, 110), bridges 112 or router devices as shown in FIG. 1. In some embodiments, the I/O devices 106 include one or more of network adapters or mass storage devices from which the computing system 100 can send and receive data for generating and transmitting advertisements with endorsements and associated news. In some embodiments, the computing system 100 is commutatively coupled with the Internet (or more generally a network 150) through the I/O devices. The memory 104 stores control programs that are executed by the processor 102. In some embodiments, control programs are stored on the network 150.

Those of ordinary skill in the art will recognize that many modifications and variations of the present disclosure may be implemented without departing from the spirit or scope of the disclosure. Thus, it is intended that the present disclosure cover the modification and variations of this disclosure provided they come within the scope of the appended claims and their equivalents. As used herein, the term “bus,” “busses,” and “bridge” refer to electrical contacts configured to transmit data or to supply low voltage power to components or devices or the present disclosure. The contacts can be electroplated finger contacts on an edge of a printed circuit board which can interdigitate with a standoff connector of a different component. The contacts can also be a MOBUS pin connector. Each electroplated finger or pin can correspond to a bit. By way of example, a connector having sixteen fingers or pins can correspond to a 16-bit data transfer connection between components. In some embodiments, the computer buses (108, 110), bridges 112 or router devices are a serial connection port, an ETHERNET connection port, a USB port, or a fiber optic connection port.

In some embodiments, the computing system 100 of the face mask 200 is connected to a computing device 106 via the network 150. In some embodiments, the computing system 100 of the face mask 200 is connected to a computing device 160 via a bus or cable connection. The computing device 160 includes a processor, 162, memory 164 connected to the processor 162, and a user interface 166 connected to the processor 162. The connection can be achieved through wireless networks or wireless connectivity, such as near-field communications or Bluetooth such that users can communicate effectively with other users 50 also using a separate face mask 200. Effective communications can be considered critical in high-stress situations, such as in a hospital operating room, a dentist office, or the like. In some embodiments, the face mask 200 can be one of many face masks 200 that connect to one another via a network 150, either ad-hoc or via a centralized server. In some embodiments, the network 150 can enable communication between face masks 200. In some embodiments, the computing device 160 is a device-enabled application or more generally a separate computing device separate from the face mask 200. The computing system 100 can receive inputs from the computing device 160, and send data between the face mask 200 and the computing device 160. In some embodiments, the computing device 160 is a mobile computing device, a personal computer, a tablet PC, or the like.

The various illustrative logics, logical blocks, modules, and engines, described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but, in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

Further, the steps or actions of a method or algorithm described in connection with the aspects disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of storage medium known in the art. An exemplary storage medium can be coupled to the processor, such that the processor can read information from, and write information to, the storage medium. In the alternative, the storage medium can be integral to the processor. Further, in some aspects, the processor and the storage medium can reside in an ASIC. Additionally, the ASIC can reside in a user terminal. In the alternative, the processor and the storage medium can reside as discrete components in a user terminal. Additionally, in some aspects, the steps and/or actions of a method or algorithm can reside as one or any combination or set of instructions on a machine readable medium and/or computer readable medium.

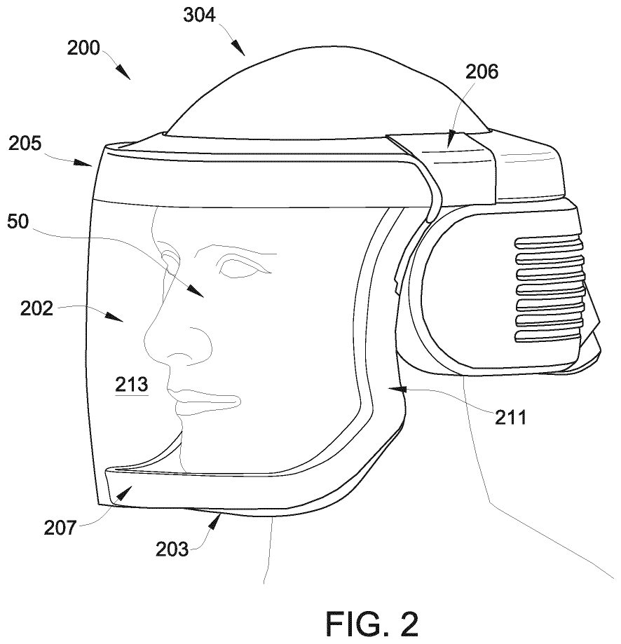

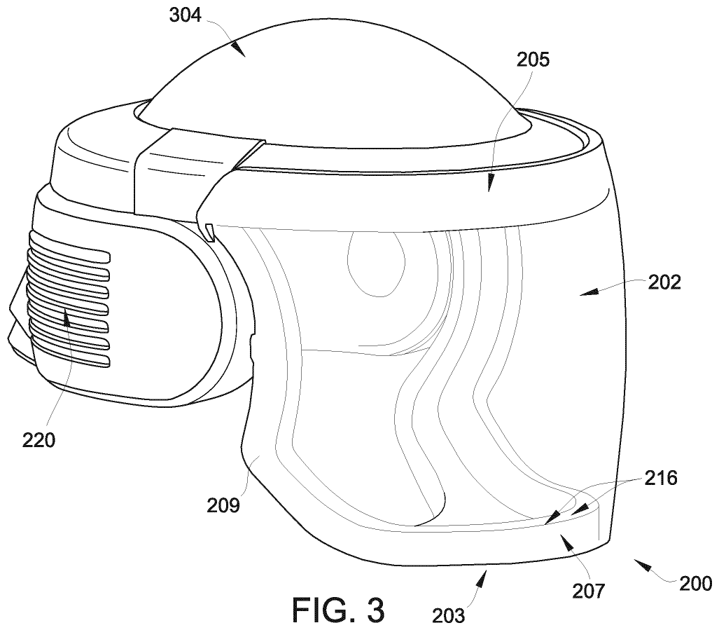

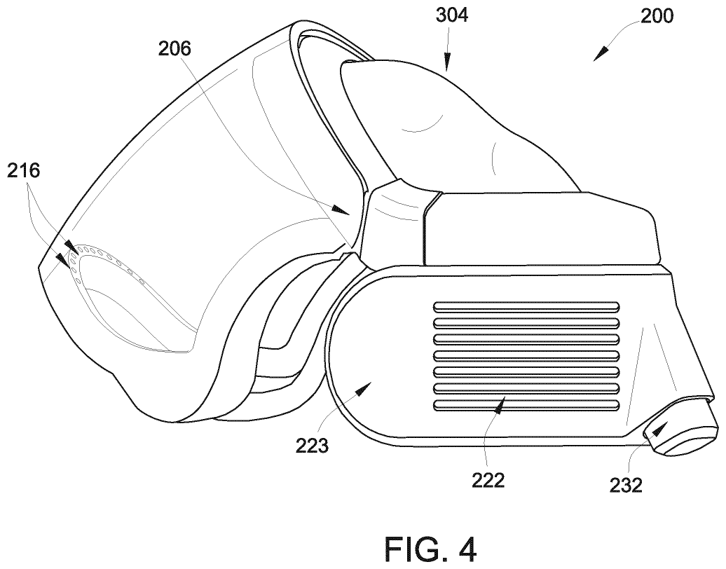

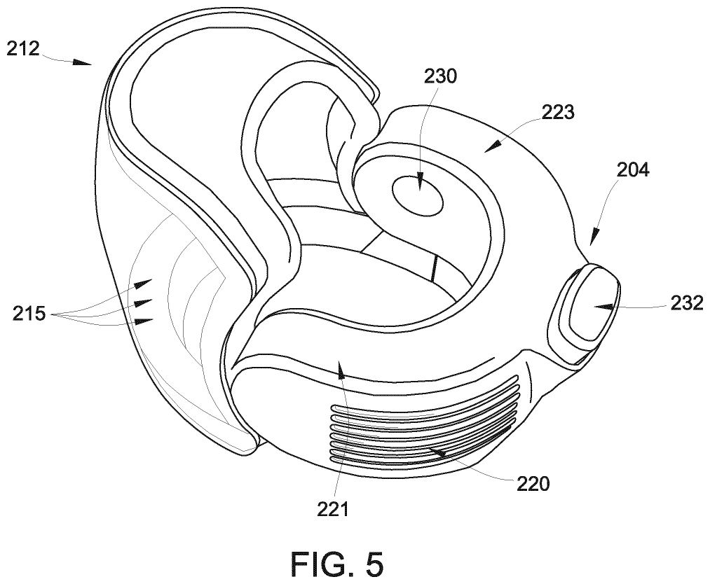

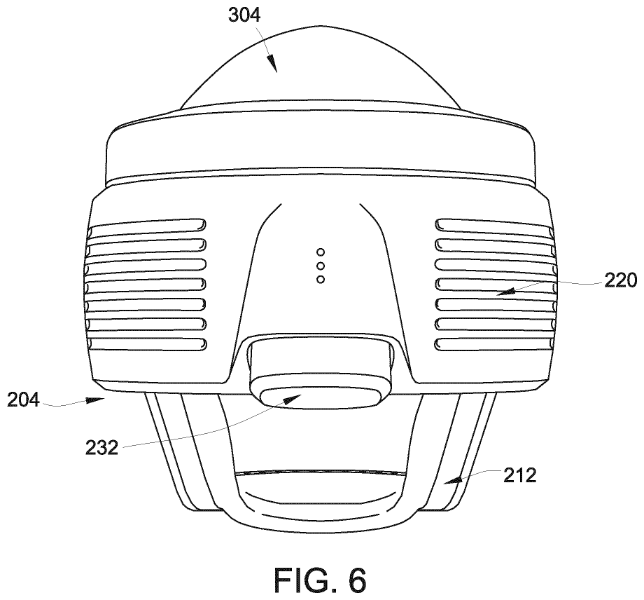

FIGS. 2 and 3 illustrate perspective views of the face mask 200, with FIG. 2 illustrating the face mask 200 in use with the user 50. FIG. 4 illustrates a side view of the face mask 200, FIG. 5 illustrates a bottom view of the face mask 200 and FIG. 6 shows a back view of the face mask 200.

The face mask 200 includes a housing 204 formed to the head of the user 50 and a ventral protective ventral visor 202 supported by a frame 203. As best shown in FIGS. 2 and 5, the housing 204 has a u-shape with first housing portion 221 and second housing portion 223 formed to the cranium of a user. Stated differently, the housing is sized and shaped to fit onto the head of a user as shown in FIG. 2. As shown in FIG. 2, in use, the housing 204 is located along the posterior of the user's head with a lateral left portion 221 and a lateral right portion 223 adjacent the user's ears. The frame 203 and the ventral visor 202 are connected such that the frame 203 and ventral visor 202 move together between an open position and a closed position. The frame 203 includes a first portion 205 and second portion 207 joined by side frame portions 209 and 211, fully enclosing the ventral visor 202 within the frame 203. Stated differently, the frame 203 includes a distal first portion 205, a proximal second portion 207 and side frame portions (209, 211), connecting the first portion 205 and second portion 207, wherein the ventral visor 202 is seated therebetween. In some embodiments, the first portion 205 and second portion 207 have generally arcuate shapes, such that, when the mask is worn, portions 205 and 207 are comfortably located proximate the forehead of the user 50 and the chin respectively while side portions (209, 211) are located adjacent the sides of the user's face. The ventral visor 202 is made of a transparent material.

In some embodiments, the ventral visor 202 is removable from the frame 203. In some embodiments the ventral visor 202 is non-removable and fluidly sealed with the frame 203 such that particles do not escape between the ventral visor 202 and the frame 203. In embodiments where the ventral visor 202 and the frame 203 are removably attached to one another, an O-ring or a sealant is positioned between the ventral visor 202 such that particles do not escape between the ventral visor 202 and the frame 203.

As best shown in FIG. 5, the second portion 207 includes a sealing member 212 integral to the second portion 207. Member 212 urges the ventral visor 202 and frame 203 towards the user's face, creating an air-tight fit. The user's face is located in an airtight chamber 213 defined by the sealing member 212 and ventral visor 202.

A plurality of first openings 215 are formed along the length of lateral left portion 221 and likewise, a plurality of second openings 216 are provided along the length of lateral right portion 223. In use, fresh ambient air can enter the air-tight chamber 213 through the plurality of first openings 215. The ambient air flows across the surface of ventral visor 202 within chamber 213. The flow of ambient air across the visor ensures that the user's vision is not impaired by fog or condensation that would otherwise form on the ventral visor 202. The respired air is drawn out of chamber 213 through the plurality of second openings 216 of the second portion 207. Stated differently, the plurality of first openings 215 are configured as an inlet and the plurality of second openings 216 are configured as an outlet. The inlet and the outlet are in fluid communication with an airtight chamber defined by the housing and the visor.

As best shown in FIG. 5, in some embodiments, speakers (230, 234) are located along an interior wall of the housing 204 and located adjacent the user's ears when worn. The speakers (230, 234) are configured to allow for communication with others when wearing the mask. Use of the speakers will be described in greater detail below.

As best shown in FIG. 4, the face mask 200 can include a flexible mid-section that allows the mask to open fully. The mask can include an elastic component 302 that urges the visor assembly towards a user's face. This feature can ensure an air-tight fit to the user. Further, a breathable textile mesh canopy 304 can secure the face mask 200 firmly on the user's head and prevent the face mask 200 from undesirably displacing from the position proximate the user's face, for example. Within housing 204 are a number of batteries, sensor and other components that will also be further described below. Battery 232 is seated in housing 204 and serves to power fans, sensors and other components requiring battery power for use.

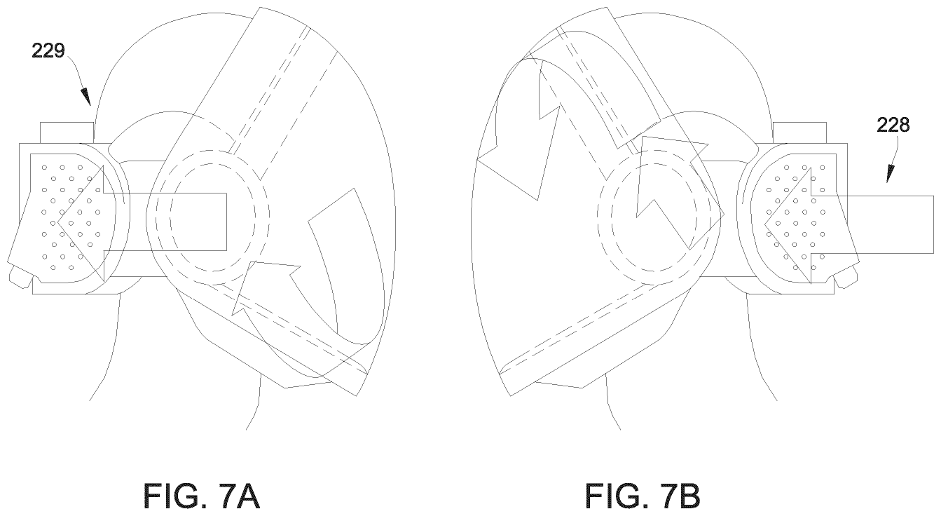

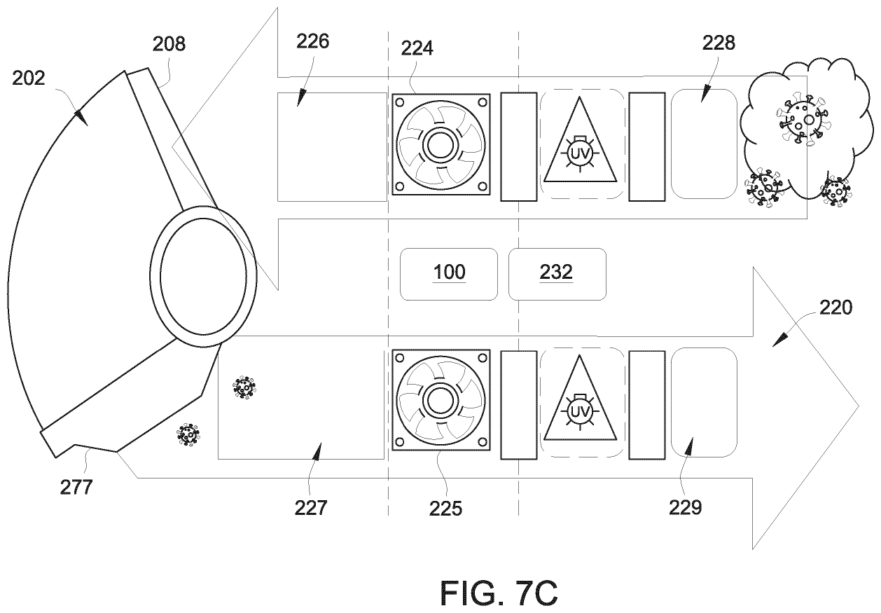

As best shown in FIGS. 4, 5, 6, 7A and 7B, an ambient air inlet 222 is positioned within the second housing portion 223 and likewise, an air outlet 220 is positioned within in first housing portion 221. The ambient air inlet 222 is in fluid communication with the chamber 213. As shown schematically in FIG. 7C, inlet and outlet fans 224 and 225, inlet and outlet air filters 228 and 229 respectively and inlet and outlet conduits 226 and 227 respectively are located in housing 204. Conduit 226 flow connects inlet 222 to the openings 215 of portion 205, and conduit 227 flow connects outlet to the openings 216 of portion 207. Filter 228 is located proximate inlet 222. As ambient air is drawn into inlet 222, contaminants, such as particulate matter, dirt, microorganism, etc, are filtered from the entrained air by inlet filter 228. The ambient air then passes through the conduit and into chamber 213 through openings 215. The air that is drawn through openings 216 by fan 225, passes through conduit 227, filter 220 and exits the housing through outlet 220. The air is filtered when it enters the housing and before it returns to the ambient environment, reducing risk of negative impact to people located proximate the user of mask 200. Further features relating to control of fan speed and airflow will be described below.

Locating the inlet and outlet at the back of the user's head rather than along the front of the mask reduces discomfort to the user that resulted from prior art masks where heavy filters are located along the front of the mask. Additionally, because the inlet and outlet and associated filters are located along the back of the user's head, the user's mouth is not blocked from view by the large filters and is in full view thereby simplifying communication. Because the filtered air exits the mask from a location along the back of the user's head, the air is not directly discharged at a person in front of the user.

In some embodiments, the face mask can be configured as a Powered Air Purifier Respirator (PAPR), that can include one or more smart features. By way of example, but not limitation, smart features can include one or more sensors or Bluetooth communication headset functionality.

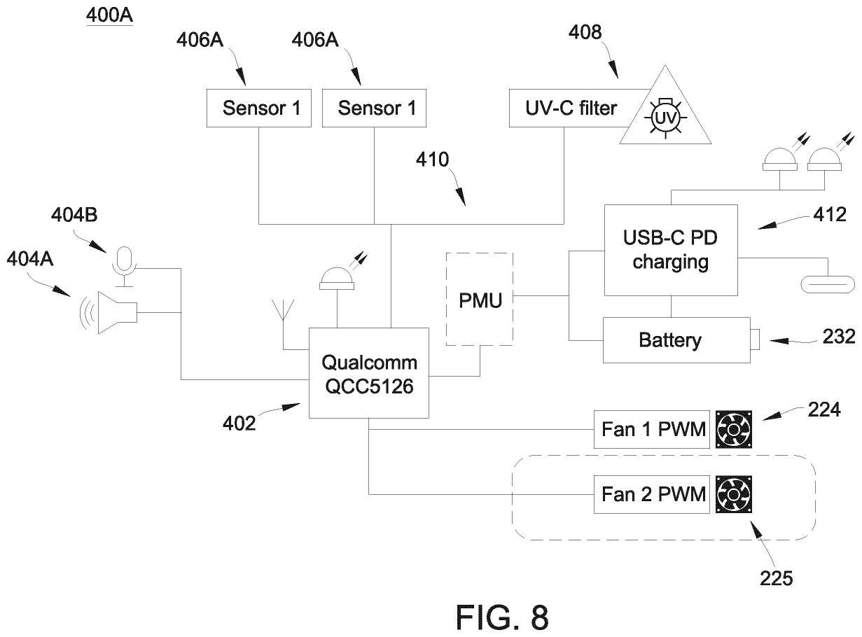

FIG. 8 illustrates an exemplary MCU system 400A overview in accordance with one or more embodiments of the face mask assembly described herein. MCU system 400A can include a built-in MCU 402. In some embodiments, the MCU can be a QCC5126 BT chip from Qualcomm, for example. It is understood that the type of chip used is not meant to be limiting. The MCU can be connected to multiple components, including but not limited to, a speaker 404A, a microphone 404B, sensors 406A and 406B, UV filter 408, PMU 410, USB charging component 412, battery 232, and one or more fans 224 and 225. Further, MCU system 400A can provide network 150 communication capabilities, such as short range or long-range wireless network 150 communications.

In some embodiments, the sensors 406A and 406B include an inlet static pressure sensor and an outlet static pressure sensor positioned proximate to the fans of the inlet 222 and the outlet 229. The inlet static pressure sensor and the outlet static pressure sensor is in communication with the MCU system 400 and the processor 102. The processor 102 is also connected to the fans of the inlet 222 and the outlet 229. The processor 102 is configured to receive static pressure data from the inlet static pressure sensor and an outlet static pressure sensor. The processor 102 is further configured to control the speed of the fans of the inlet 222 and the outlet 229.

In some embodiments, during a calibration phase, the processor 102 is configured to collect static pressure data as the user is breathing during a non-strenuous activity over a period of time and store the data in the memory 164. The processor 102 is the configured to determine an upper limit of inhale and exhale static pressure data for the calibration phase. During a detection phase, if the processor determines that the upper limit is exceeded, the processor 102 is configured to increase a fan speed of either or both of the fans of the inlet 222 and the outlet 229. If the static pressure of the inlet is greater than the upper limit stored in memory, the processor 102 is configured to increase the fan speed of the inlet 222. Likewise, if the static pressure of the outlet is greater than the upper limit stored in memory, the processor 102 is configured to increase the fan speed of the outlet 229. Thus, during a strenuous activity, the face mask 200 can aid in breathing of the user. The calibration can be done for a first user and for a second user, and the limits can be stored in memory.

MCU system 400A can include one or more communication components, such as a speaker 404A and a microphone 404B. The communication components, in at least one example, can enable a user of the face mask to have clear communication with surrounding individuals. In some embodiments, surrounding individuals can also be wearing a similar type of face mask. Alternatively, the user can communicate with another person by other means, such as a phone call over Bluetooth, or the like. The speaker and microphone may comprise built-in components that enhance voice communication when the face mask is worn and in operation. In one implementation example, when a user of the face mask speaks, the microphone can be enabled to pick up the voice with the mic and then play back the captured voice audio via an external speaker. In another embodiment, the external voice enhancement playback provides one or more enhancements to the user's voice such that the person, or persons, being spoken to perceive the voice of the mask user as if there were no mask being worn at all. The microphone can be an integrated microphone that enables the external voice enhancement. Additionally, the integrated components described herein can further provide phone calls in handsfree mode. The MCU system can be connected to multiple audio sources, such as MCU system sounds, Bluetooth audio from a headset microphone, or a mic input from an internal microphone, for example. The MCU system can be connected to multiple audio end points, such as an external speaker, a headphone speaker, or the like.

In some embodiments, MCU system 400A can include two DSP cores for audio and two application processors, for example. In one example, a first application processor can handle a Bluetooth stack and a second application can handle user code. The face mask assembly can support different Bluetooth protocols, such as BLE or BT Classic, for example. Further, MCU system 400A can include an antenna to provide network 150 connectivity. For example, MCU system 400A can include an integrated 2.4 GHz antenna for Bluetooth connectivity, for example. Code and algorithms can be executed from an external Serial Flash. Further, MCU system 400A can include an audio hub connected to multiple components, such as earpieces, microphones, speakers, throat microphones, or the like. Further, MCU system 400A can include Bluetooth audio profiles, such as A2DP, HFP, HSP, and/or BLE services. Even further, MCU 400A can control power distribution to other subsystems. Other subsystems can include, but is not limited to, an inertial measurement unit (IMU), a user interface comprising buttons, LEDs, etc., and external flash memory.

MCU system 400A can include multiple sensors, such as Sensors 406A and 406B. Different types of sensors can be used depending on functionality needs of the smart mask. Sensors 406A and 406B can connect to MCU 402 via a bus, such as a I2C bus, for example. Implementation can be realized using software, hardware, or a combination thereof. In some embodiments, sensor 406A can be an ambient light sensor (ALS). An ALS can be used measure surrounding light. The measurement can be used to adapt the brightness of one or more LEDs within the face mask user's field of view. Further, brightness adjustment can provide a constant light intensity at a comfortable level. Another type of sensor utilized by the face mask assembly can be a temperature sensor. A temperature sensor can, for example, monitor the temperature of the face mask user. Data gathered by the one or more sensors can be collected and transmitted to one or more locations. For example, the gathered data can be transmitted over a network 150 to a remote device, remote storage, or the like.

In some embodiments, the mask assembly can include a UV filter 408, such as a UV-C filter, or the like. For example, UV light with a wavelength between 100-280 nm can be accepted through field emission in addition to LED or other traditional methods. The utilization of UV-C along with the HEPA filter is that the UV-C can help prolong the life of the HEPA filter by killing the bacteria, virus or others caught by the HEPA filter, thus reducing the need to frequently replace the HEPA filter.

One or more biosensors can be implemented for use with the face mask 200. Biosensors can provide intelligent controlling of the UV-C intensity and air flow in places where at least one biosensor detects that there is higher presence of biomolecule, a biological structure or a microorganisms; and the converse of intelligently reducing the UV-C intensity and airflow when the user/user is in a cleaner environment.

An exemplary system of the mask can include USB-C charging 412. In some embodiments, an integrated battery 232 can be charged via USB-C. The USB-C connector can charge battery 232 without needing to remove the battery from the mask or possibly interrupting the operation of the mask. In some embodiments, a power bank (not shown) can be utilized if the connected battery is running low on charge. A USB port can be utilized that supports data communication for FW upgrade and of logging during development. The connector can support both USB 2.0 full speed (12 Mbps) and high speed (480 Mbps), for example.

Battery 232 can be a custom battery pack (possibly made by 18650 batteries) with a target capacity of at least 30 Wh. The capacity can be set to support the use case to use the respiratory function for up to 8 h with one fan running at nominal speed. battery packs are replaceable during the use of the Smart mask by snapping loose the battery and insert a fully charged battery on the fly. The smart mask will temporarily shut down while no power is available. When a charged battery is inserted the smart mask will automatically power on again.

During normal operation of the face mask 200, the user can initiate operation of the mask by depressing a button, such as an on-off button, located on the mask assembly. In some embodiments, depression can need to occur for at least three seconds to initiate powering on procedures. Additionally, or alternatively, on/off control of the face mask can be performed via a remote application. In some embodiments, a user can interact with one or more settings via a mobile application on the user's mobile device that communicates with the mask. Settings and other information shared between the mobile app and mask can include, but are not limited to, running time, filter settings, battery levels, air respiration settings, or the like.

In some embodiments, the mask can include one or more physical volume buttons ergonomically placed on the mechanics near the headphones for easy access. Additionally, or alternatively, volume control can be adjusted via the remote application. Further, action buttons can be provided wherein the action button can cause multiple functionalities. A reset button can be provided for resetting the system.

In some embodiments, the mask can include one or more respiratory systems, as illustrated by FIGS. 4B and 4C. The respiratory system aspects can include one or more filters. The one or more filters can be standard filters, such as STANAG, EN-148, or the like. The mask can also include a standard filter having threading on both in and an optional out filter.

In some embodiments, the mask can include two fans 224 and 225 to promote the flow of air. For example, a first fan 224 can promote airflow into the mask as an inlet fan via inlet 228. A second fan 225 can be used to operate as an outlet fan and via outlet 229. Additionally, each fan can include a filter, such as filters 226 and 227. The filters can be reusable or disposable. In some embodiments, the filter and fan assemblies can be removable for easy cleaning or replacement. Additionally, or alternatively, if only an incoming filter/fan is utilized, the air outlet can have a check valve to ensure no unfiltered air is passed in through the outlet.

The airflow can flow from the intake to the outlet as shown in FIGS. 4B and 4C. Air can be drawn in on the back left side, guided to the top of the mask and down on the inside of the visor to prevent fog on the glass on the way to the mouth of the user. The air can then be guided out in the lower half, on the right-side and then out through the outlet filter on the back right side. The inlet filter and the outlet filter can be mounted at an angle relative each other that ensures no re-circulation of air occurs from the outlet to the inlet. It is understood that the intake and outlet can by swapped and the example shown is in no way meant to be limiting.

Low noise level by the smart mask components provide a good user experience. The fans being controlled by the MCU are adjusted dynamically to keep the fan rpm as low as possible while keeping the air flow within the face mask within the specified limited. The MCU monitors the air flow via the one or more sensors built-in to the assembly. The respiratory fans can consume >7 W when both fans are running at full speed with particle and UV-C filters on.

MCU system 400A can also control, alter, and adjust speeds of fans 224 and 225 based on information gathered by sensors of the face mask assembly, such as sensors 406A and 406B. In some embodiments, a condition of the user can be detected by one or more sensors or biosensors with respect to the user's breathing rate, heart rate, respiratory condition, body temperature, blood pressure, oxygen levels, or the like. Other types of data can be sensed, such as weather conditions, internal temperatures within the mask assembly, or the like. Based on this or other types of data acquired by the sensors described herein and above, the MCU 402 can issue one or more commands to adjust fans 224 and 225, as needed. Additionally, or alternatively, a mobile device user can alter fan speeds based on gathered data viewed on a user interface. Even further, a user can customize desired fan speed settings based on one or more sensor readings while the face mask is in use. The user can also be provided with one or more presets or default settings.

In some embodiments, the face mask 200 can provide protection to a user 50 of the face mask 200 from harmful contaminants found in the surrounding environment. In some embodiments, the face mask 200 can protect individuals within a vicinity of the user 50 of the face mask 200 from harmful contaminants that can be expressed into the air by the user 50. For example, if the user 50 is a carrier of a contagious disease, such as the seasonal flu, the face mask 200 can provide adequate filtering of exhaled air from the user 50. In some embodiments, the face mask 200 can be worn in shared spaces, such as indoor environments, an office, a restaurant, an elevator, or the like. In some embodiments, the face mask 200 can be worn in outdoor environments, such as sporting events, concerts, family gatherings, or the like.

Mask 200 can be an active face mask that provides a full-face powered air purifier respirator. The mask 200 can provide active fan powered airflow to the mask user via two-way air filtering. Further, the user of the mask 200 can be provided with smart audio functionality. Mask 200 can include a single full-face cavity that provides full-face visibility. All internal components, such as electronics, filters, and fans are placed in the rear of the mask assembly. Such placement provides for balance and providing the air exhaust to the rear of the user.

Mask 200 can also include an exchangeable battery pack having a power indicator. The mask can include integrated earphones with noise cancellation and hear-through functionality. Additionally, or alternatively, mask 200 can be equipped with external speakers thereby providing speech amplification.

In some embodiments, mask 200 can include removable and washable internal cushioning for a personalized fit and provide comfort for the user. Air holes can be provided along the top of the visor of the mask assembly. Fresh air can flow from the air holes and enter the full-face cavity.

It is appreciated that the exemplary computing system 100 of FIG. 1 is merely illustrative of a computing environment in which the herein described systems and methods can operate, and thus does not limit the implementation of the herein described systems and methods in computing environments having differing components and configurations. That is, the inventive concepts described herein can be implemented in various computing environments using various components and configurations.

Those of skill in the art will appreciate that the herein described apparatuses, engines, devices, systems and methods are susceptible to various modifications and alternative constructions. There is no intention to limit the scope of the disclosure to the specific constructions described herein. Rather, the herein described systems and methods are intended to cover all modifications, alternative constructions, and equivalents falling within the scope and spirit of the disclosure, any appended claims and any equivalents thereto.

In the foregoing detailed description, it can be that various features are grouped together in individual embodiments for the purpose of brevity in the disclosure. This method of disclosure is not to be interpreted as reflecting an intention that any subsequently claimed embodiments require more features than are expressly recited.

Further, the descriptions of the disclosure are provided to enable any person skilled in the art to make or use the disclosed embodiments. Various modifications to the disclosure will be readily apparent to those skilled in the art, and the generic principles defined herein can be applied to other variations without departing from the spirit or scope of the disclosure. Thus, the disclosure is not intended to be limited to the examples and designs described herein, but rather is to be accorded the widest scope consistent with the principles and novel features disclosed herein.

1. A protective face mask assembly, comprising:
a housing sized and configured to fit onto a head of a user, the housing having a lateral left portion and a lateral right portion;
a visor supported by a frame, the visor and frame forming a sealed structure;
a chamber defined by the housing and the frame;
a plurality of first openings formed along the left lateral portion configured as an air inlet; and,
a plurality of second openings formed along the right lateral portion configured as an air outlet;
wherein the plurality of first openings and the plurality of second openings are in fluid communication with the chamber.

2. The assembly of claim 1, further comprising an inlet filter proximate the plurality of first openings.

3. The assembly of claim 1, further comprising an outlet filter proximate the plurality of second openings.

4. The assembly of claim 2, wherein the filter is a UV filter.

5. The assembly of claim 1, wherein air flows from the inlet across a surface of the visor within the chamber to the outlet, wherein the chamber is airtight when worn by the user.

6. The assembly of claim 1, wherein the frame is movable between a closed position and an open position.

7. The assembly of claim 6 further comprising a flexible midsection connecting the frame to the housing.

8. The assembly of claim 1, wherein the visor is supported by the frame.

9. The assembly of claim 8, wherein the frame includes a sealing member integral to the frame configured to urge the visor and the frame towards a face of the user creating an air-tight fit.

10. The assembly of claim 1, wherein the visor is removable from the frame.

11. The assembly of claim 10, wherein a sealant is positioned between the visor and the frame.

12. The assembly of claim 1 further comprising a textile mesh canopy providing a secure fit of the assembly to the head of the user.

13. The assembly of claim 1 further comprising speakers located along an interior wall of the housing.

14. The assembly of claim 1, wherein the air inlet includes a first fan, and the outlet includes a second fan.

15. The assembly of claim 14, wherein the face mask is configured as a Powered Air Purifier Respirator, wherein an inlet static pressure sensor and an outlet static pressure sensor positioned proximate to the first fan and the second fan, wherein the inlet static pressure sensor and the outlet static pressure sensor is in communication with a processor, the processor configured to receive static pressure data from the inlet static pressure sensor and an outlet static pressure sensor, wherein the processor is further configured to control the speed of the fans of the inlet and the outlet.

16. The assembly of claim 1 further comprising a processor and memory.

17. The assembly of claim 16, wherein the processor is configured to connect to a smart device by use of a network.

18. The assembly of claim 17, wherein the processor is connected to one or more sensors including one or more of an ambient light sensor, a temperature sensor, or a biosensor.

19. The assembly of claim 18, wherein the processor is configured to receive data from the one or more sensors and transmit data to the smart device via the network

20. The assembly of claim 1 further comprising a check valve positioned between the outlet and the chamber.
